(19) United States
(12) Patent Application Publication
(10) Pub. No.: US 2009/0023744 A1
(43) Pub. Date: Jan. 22, 2009

(54) COMBINATION THERAPY FOR DEPRESSION

(75) Inventor: Maurizio Fava, Newtonville, MA (US)

Correspondence Address: WOLF GREENFIELD & SACKS, P.C., 600 ATLANTIC AVENUE, BOSTON, MA, 02210-2206, US

(73) Assignee: The General Hospital Corporation, Boston, MA (US)

(21) Appl. No.: 12/140,498

(22) Filed: Jun. 17, 2008

Related U.S. Application Data
(60) Provisional application No. 60/936,110, filed on Jun. 18, 2007.

(51) Int. Cl. A61K 31/496; A61K 31/137; A61P 25/24

(52) U.S. Cl. 514/252.15

(57) ABSTRACT

A combination therapy for treating depressive disorders is provided herein. The combination therapy comprises administering an effective amount of bupropion or its metabolites together with at least one serotonin 5-HT_{1A} partial agonist or its metabolites to a patient in need of treatment of the depressive disorder. Pharmaceutical formulations, including packaged pharmaceutical formulations, comprising this combination are also provided herein.

### RELATED APPLICATION

This application claims the benefit under 35 U.S.C. § 119(e) of U.S. provisional application 60/936,110, filed Jun. 18, 2007, the entire disclosure of which is incorporated herein by reference.

### BACKGROUND OF THE INVENTION

Depressive disorders make up a class of psychiatric disorders characterized by an pathological depressive state and include, for example, major depressive disorder (MDD), bipolar disorder, dysthymic disorder and minor depressive disorder. The prevalence of depressive disorders is high; the lifetime prevalence of major depressive disorder in the United States is about 15%.

Numerous therapies are available for treating patients suffering from depressive disorders. However, a large percent of those treated with conventional drug therapies experience only partial relief of their symptoms or do not respond at all to such treatments. The largest treatment study ever conducted in depression, the Sequenced Treatment Alternatives to Relieve Depression (STAR*D) trial, has shown that only one in three patients with major depressive disorder achieve remission with an adequate trial of selective serotonin reuptake inhibitors (SSRIs), currently used as standard treatments of MDD and generally considered by psychiatrists and primary care physicians to be effective, well-tolerated and easily administered. Therefore, SSRIs are associated with undesirable features, such as a level of non-responsiveness estimated to be as high as 30%, and a high incidence of side effects including sexual dysfunction and weight gain.

Thus, there is a need for pharmacological therapies that can be used to treat depressive disorders to enhance the chances of remission or in patients who do not respond to currently available therapies or who have undesirable side effects from currently available therapies.

### SUMMARY OF THE INVENTION

It has been discovered that administering a combination of agents, bupropion and (at least one) serotonin 5-HT_{1A} partial agonist, such as azapirones, preferably buspirone, provides surprising, synergistic effects for treating depressive disorders in an individual. Pharmacologically active metabolites, salts, solvates and racemates of bupropion and pharmacologically active metabolites of (at least one) serotonin 5-HT_{1A} partial agonist, such as azapirones, preferably buspirone, also can be used in the combination therapies of the invention. Such an approach—combination or co-administration of the two types of agents—is particularly useful for treating individuals suffering from a depressive disorder who do not respond to or are resistant to currently-available therapies and is also useful for improving the efficacy and/or reducing the side effects of currently-available depressive disorder therapies for individuals who do respond to such therapies.

Bupropion is an FDA-approved antidepressant that affects dopamine and norepinephrine neurotransmission, and thus has not been combined with the serotonergic anti-anxiety drug buspirone for treatment of depressive disorders. Buspirone is used only to further the action of serotonergic antidepressants (such as the SSRIs and the serotonin-norpeinephrine reuptake inibitors—SNRIs) in treatment of depressive disorders, with the goal of further enhancing serotonin neurotransmission. Without wishing to be held to any particular neurobiological theory, it is applicant's view that these two drugs are complimentary in that they affect different neurotransmitter pathways, and thereby the combination of agents provides advantageous and unexpected synergistic effects.

The combination of agents has unexpected results and advantages. As described herein, low doses of buspirone and bupropion were used effectively to treat MDD, thus providing a basis for the view that there is synergy in administering the combination of agents that may permit lower dosing of each agent (relative to presently approved, recommended doses used by clinicians) to get effective treatment. This provides potentially dramatic tolerability and efficacy advantages over other anti-depressants.

One of the major challenges in treatment of depressive disorders is that 40% of patients experience sexual dysfunction and 20% of patients experience weight gain using current therapies. A further advantage of the combination of agents is that neither of these agents causes weight gain or sexual dysfunction.

According to one aspect of the invention, methods of treating a depressive disorder in an individual are provided. The methods include administering to the individual an amount of (1) bupropion and/or a salt, solvate, metabolite or racemate thereof, and an amount of (2) at least one serotonin 5-HT_{1A} partial agonist and/or a salt, solvate, metabolite or racemate thereof, wherein the combined amount of (1) and the amount of (2) is effective for treatment of the depressive disorder.

In some embodiments, the serotonin 5-HT_{1A} partial agonist is an azapirone and/or a salt, solvate, metabolite or racemate thereof. Preferably the azapirone is buspirone, gepirone, ipsapirone, tandospirone or zalospirone, and/or a salt, solvate, metabolite or racemate thereof. More preferably, azapirone is buspirone and/or a salt, solvate, metabolite or racemate thereof. Most preferably, the azapirone is buspirone.

In a particularly preferred embodiment, bupropion and buspirone are administered to the individual.

In certain embodiments, the depressive disorder is major depressive disorder, dysthymic disorder, minor depressive disorder, or bipolar disorder.

In some embodiments, the treatment includes co-administering the amount of (1) and the amount of (2). Preferably the amount of (1) and the amount of (2) are in a single formulation or unit dosage form. In other embodiments, the methods further include administering a formulation or unit dosage form that comprises an amount of (1) but no amount of (2) and/or a formulation or unit dosage form that comprises an amount of (2) but no amount of (1). In still other embodiments, the amount of (1) and the amount of (2) are in a separate formulations or unit dosage forms.

In the foregoing methods, the treatment can include administering the amount of (1) and the amount of (2) at substantially the same time or administering the amount of (1) and the amount of (2) at different times.

In some embodiments of the foregoing methods, the amount of (1) and/or the amount of (2) is administered at dosages that would not be effective when one or both of (1) and (2) is administered alone, but which amounts are effective in combination.

According to another aspect of the invention, pharmaceutical formulations are provided. The pharmaceutical formulations include an amount of (1) bupropion and/or a salt, solvate, metabolite or racemate thereof, and an amount of (2) at least one serotonin 5-HT_{1A} partial agonist and/or a salt, solvate, metabolite or racemate thereof, wherein the combined amount of (1) and (2) is effective for treatment of depressive disorder.

In some embodiments, the serotonin 5-HT_{1A} partial agonist is an azapirone and/or a salt, solvate, metabolite or racemate thereof. Preferably the azapirone is buspirone, gepirone, ipsapirone, tandospirone or zalospirone, and/or a salt, solvate, metabolite or racemate thereof. More preferably, azapirone is buspirone and/or a salt, solvate, metabolite or racemate thereof. Most preferably, the azapirone is buspirone.

In certain embodiments, the amount of (1) and the amount of (2) are in a single formulation or unit dosage form. The formulation or unit dosage form can be an oral formulation or unit dosage form. Preferably the oral formulation or unit dosage form is a liquid, a syrup, a tablet, a capsule, a powder, a sprinkle, a chewtab, or a dissolvable disc.

In some embodiments of the foregoing pharmaceutical formulations, the amount of (1) and/or the amount of (2) would not be effective when one or both of (1) and (2) is administered alone, but which amounts are effective in combination.

According to a further aspect of the invention, pharmaceutical products are provided. The pharmaceutical products include a first formulation or unit dosage form comprising an amount of (1) bupropion and/or a salt, solvate, metabolite or racemate thereof, and an amount of (2) at least one serotonin 5-HT_{1A} partial agonist and/or a salt, solvate, metabolite or racemate thereof, and a second formulation or unit dosage form comprising an amount of (1) but not (2) or an amount of (2) but not (1). The combination of the first formulation or unit dosage form and the second formulation or unit dosage form is effective for treatment of a depressive disorder.

According to still another aspect of the invention, additional pharmaceutical products are provided. The pharmaceutical products include a first formulation or unit dosage form comprising an amount of bupropion and/or a salt, solvate, metabolite or racemate thereof, and a second formulation or unit dosage form comprising an amount of at least one serotonin 5-HT_{1A} partial agonist and/or a salt, solvate, metabolite or racemate thereof. The combination of the first formulation or unit dosage form and the second formulation or unit dosage form is effective for treatment of a depressive disorder.

In some embodiments of the pharmaceutical products, the serotonin 5-HT_{1A} partial agonist is an azapirone and/or a salt, solvate, metabolite or racemate thereof. Preferably the azapirone is buspirone, gepirone, ipsapirone, tandospirone or zalospirone, and/or a salt, solvate, metabolite or racemate thereof. More preferably, azapirone is buspirone and/or a salt, solvate, metabolite or racemate thereof. Most preferably, the azapirone is buspirone.

In certain embodiments, the formulation or unit dosage form(s) is an oral formulation or unit dosage form. Preferably the oral formulation or unit dosage form is a liquid, a syrup, a tablet, a capsule, a powder, a sprinkle, a chewtab, or a dissolvable disc.

In some embodiments of the foregoing pharmaceutical products, the amount of (1) and/or the amount of (2) would not be effective when one or both of (1) and (2) is administered alone, but which amounts are effective in combination.

Use of the combination of agents for the preparation of a medicament for the treatment of depressive disorders also is provided.

These and other embodiments of the invention, as well as additional inventive features, will be apparent from the description of the invention provided herein.

### DETAILED DESCRIPTION OF THE INVENTION

Prior to setting forth the invention in detail, it may be helpful to provide definitions of certain terms to be used herein. Compounds of the present invention are described using standard nomenclature. Unless defined otherwise, all technical and scientific terms used herein have the same meaning as is commonly understood by one of skill in the art to which this invention belongs.

The invention provides for a combination of therapeutic agents and administration of the combination of agents to treat depressive disorders. As used herein, a “combination of agents” and similar terms refer to a combination of two types of agents: (1) bupropion and/or pharmacologically active metabolites, salts, solvates and racemates of bupropion and (2) at least one serotonin 5-HT_{1A} partial agonist, such as azapirones, preferably buspirone, and/or pharmacologically active metabolites, salts, solvates and racemates of serotonin 5-HT_{1A} partial agonist(s), such as azapirones, preferably buspirone. Use of racemic mixtures of the individual agents also is provided. Pharmacologically active metabolites include those that are inactive but converted into pharmacologically active forms in the body after administration.

Bupropion is approved for treatment of depression. Bupropion affects dopamine and norepinephrine neurotransmission. Buspirone is approved for treatment of anxiety. Is also used to augment treatment with SSRIs in the treatment of depression due to its effects on serotonin type 1A receptors.

Bupropion is designated as (±)-1-(3-chlorophenyl)-2-[(1,1-dimethylethyl)amino]-1-propanone (see U.S. Pat. Nos. 3,819,706 and 3,885,046), and preferably is administered as the hydrochloride salt, which is marketed as WELLBUTRIN, ZYBAN, BUDEPRION and BUPROBAN. Metabolites of bupropion include hydroxybupropion, the erythro- and threo-amino alcohols of bupropion, the erythro-amino diol of bupropion, and morpholinol metabolites of bupropion, (±)-(2R*,3R*)-2-(3-chlorophenyl)-3,5,5-trimethyl-2-morpholinol, (−)-(2R*,3R*)-2-(3-chlorophenyl)-3,5,5-trimethyl-2-morpholinol, and (+)-(2S,3S)-2-(3-chlorophenyl)-3,5,5-trimethyl-2-morpholinol, which latter molecule is known by its common name of radafaxine. Other bupropion metabolites are described in U.S. Pat. No. 6,342,496 and U.S. Pat. No. 6,337,328, including: 2-(3-chlorophenyl)-2-hydroxy-3,5,5-trimethyl-morpholinol, 1-(3-chlorophenyl)-2-[(1,1-dimethylethanol)amino]-1-propanol, 1-(3-chlorophenyl)-2-[(1,1-dimethylethanol)amino]-1-propanone, (R,R)-2-(3-chlorophenyl)-2-hydroxy-3,5,5-trimethyl-morpholinol; (S,R)-2-(3-chlorophenyl)-2-hydroxy-3,5,5-trimethyl-morpholinol; (S,S)-2-(3-chlorophenyl)-2-hydroxy-3,5,5-trimethyl-morpholinol (i.e., (S,S)-hydroxybupropion); (R,S)-2-(3-chlorophenyl)-2-hydroxy-3,5,5-trimethyl-morpholinol; (R,R)-1-(3-chlorophenyl)-2-[(1,1-dimethylethanol)amino]-1-propanol; (S,R)-1-(3-chlorophenyl)-2-[(1,1-dimethylethanol)amino]-1-propanol; (S,S)-1-(3-chlorophenyl)-2-[(1,1-dimethylethanol)amino]-1-propanol; (R,S)-1-(3-chlorophenyl)-2-[(1,1-dimethylethanol)amino]-1-propanol; (R)-1-(3-chlorophenyl)-2-[(1,1-dimethylethanol)amino]-1-propanone; and (S)-1-(3-chlorophenyl)-2-[(1,1-dimethylethanol)amino]-1-propanone. Optically pure stereoisomers are described, for example, in U.S. Pat. No. 6,110,973.

Buspirone is designated as 8-[4-[4-(2-pyrimidinyl)-1-piperazinyl) butyl)-8-azaspiro[4,5]decane-7,9-dione (see U.S. Pat. No. 3,717,634), and preferably is administered as the hydrochloride salt, which is marketed as BUSPAR, ANSIAL, ANSICED, ANXIRON, AXOREN, BESPAR, BUSPIMEN, BUSPINOL, BUSPISAL, NAROL, SPITOMIN and BUSPIREX. Metabolites of buspirone include 1-(2-Pyrimidinyl)-piperazine (1-PP); BMY 28674 (6-Hydroxy-8-[4-[4-(2-pyrimidinyl)-piperazinyl]-butyl]-8-azaspiro[4,5]-7,9-dione; see EP 1 248 622); hydroxy-buspirone, e.g. 6-hydroxy-buspirone (6OHB); hydroxy-buspirone glucuronide; dihydroxy-buspirone; and dihydroxy-buspirone glucuronide. Stereoisomers of buspirone metabolites include the R-stereoisomer of 6-hydroxy-buspirone (U.S. Pat. No. 6,686,361) and the S-stereoisomer of 6-hydroxy-buspirone (U.S. Pat. No. 6,821,976). Various formulations of buspirone are known in the art; for example, see U.S. Pat. No. 5,431,922, U.S. Pat. No. 6,268,368, and references described therein.

In addition to buspirone, other azapirones useful in accordance with the invention include gepirone, ipsapirone, tandospirone and zalospirone.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Agents may contain one or more asymmetric elements such as stereogenic centers or stereogenic axes e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms; all isomeric forms of the compounds are included in the present invention. In these situations, the single enantiomers (optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Unless otherwise specified, or clearly indicated by the text, reference to compounds useful in the combination therapy of the invention includes both the free base of the compounds, and all pharmaceutically acceptable salts of the compounds. A preferred buspirone salt is the monohydrochloride salt. A preferred bupropion salt is the hydrochloride salt. The terms “buspirone or its salts”, “bupropion or its salts” and the like, indicate the pharmaceutically acceptable salts of buspirone and bupropion, respectively.

The term “pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

An “effective amount” of a combination of agents (e.g., bupropion and buspirone) is an amount sufficient to provide an observable improvement over the baseline clinically observable signs and symptoms of the depressive disorder treated with the combination.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “releasable form” is meant to include instant release, immediate-release, controlled-release, and sustained-release forms.

By “instant-release” is meant a dosage form designed to ensure rapid dissolution of the active agent by modifying the normal crystal form of the active agent to obtain a more rapid dissolution.

By “immediate-release” is meant a conventional or non-modified release form in which greater then or equal to about 50% or more preferably about 75% of the active agents is released within two hours of administration, preferably within one hour of administration.

By “controlled-release” is meant a dosage form in which the release of active agents is controlled or modified over a period of time. Controlled can mean, for example, sustained, delayed or pulsed-release at a particular time. Alternatively, controlled can mean that the release of active agents is extended for longer than it would be in an immediate-release dosage (e.g., over one or several hours).

By “sustained-release” or “extended-release” is meant to include the release of active agents at such a rate that blood (e.g., plasma) levels are maintained within a therapeutic range but below toxic levels for at least about 8 hours, preferably at least about 12 hours, more preferably about 24 hours after administration at steady-state. The term “steady-state” means that a plasma level for a given active agent or combination of active agents, has been achieved and which is maintained with subsequent doses of the active agent(s) at a level which is at or above the minimum effective therapeutic level and is below the minimum toxic plasma level for a given active agent(s).

By “water-soluble” agent is meant an agent, or combination of active agents, that are at least slightly water-soluble (for example, about 1 to about 10 mg/ml at 25° C.). Preferably, all active agents are moderately water-soluble (for example, less than about 100 mg/ml at 25° C.), or highly water-soluble (for example, greater than about 100 mg/ml at 25° C.).

By “water-insoluble” or “poorly soluble” active agent, it is meant an agent having a water solubility of less than 1 mg/ml, and in some cases even less than 0.1 mg/ml.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

### Methods of Treatment

The invention provides a method of treating a depressive disorder, such as major depressive disorder, in an individual by administering to the individual a combination of agents as defined herein, such as bupropion and (at least one) serotonin 5-HT_{1A} partial agonist, preferably an azapirone, more preferably buspirone.

The methods of the present invention are useful for the treatment of a variety of depressive disorders. Depressive disorders, also called mood disorders, are mental disorders characterized by pathological, persistent or episodic exaggeration of a mood state. Depressive disorders include, but are not limited to, the following disorders: Depressive Disorders: e.g., Major Depressive Disorder—single episode; Major Depressive Disorder—recurrent, and Depressive Disorder—Not Otherwise Specified (NOS), Dysthymic Disorder, depressive neurosis, depressive reaction, postpartum depression, premenstrual syndrome, and neurotic depression; Bipolar Disorders: e.g. Bipolar I Disorder, Bipolar II Disorder, and Cyclothymic Disorder; and Mood Disorders due to a general medical condition: e.g. with Depressive, Manic, or Mixed Features.

Subtypes of depressive disorders also are treatable with the methods and combinations of agents of the present invention. Such subtypes include atypical depression, anxious depression and depression with melancholic features. Without wishing to be held to any particular theory about the mechanism of action of the treatment with the combination of agents, it is believed that the effectiveness for depressive disorders such as atypical depression may be due to the effects of bupropion on dopamine, the effectiveness for depressive disorders such as anxious depression may be due to the antianxiety effects of buspirone, and the effectiveness for depressive disorders such as depression with melancholic features may be due to the effects of the combination of agents (e.g., bupropion and buspirone) on three neurotransmitter systems (norepinephrine, serotonin, and dopamine), as agents affecting two of these systems have shown greater efficacy in this subtype. The methods and combinations of agents of the present invention are also useful to treat anxiety disorders, such as obsessive-compulsive disorder, generalized anxiety disorder, social anxiety disorder or phobias, and post-traumatic stress disorder.

In some embodiments, the individual to be treated (i.e., patient) is determined to be non-responsive or resistant to one or more depression therapies, e.g., SSRIs. In other embodiments, the individual to be treated was responsive to depression therapy, but the therapy has side effects, which may be of such severity that the individual reduces or stops all or part of the existing therapy.

Methods of treating a depressive disorder by administering an effective amount of bupropion and (at least one) serotonin 5-HT_{1A} partial agonist, such as azapirones, preferably buspirone, to an individual having a depressive disorder, such as major depressive disorder, dysthymic disorder, bipolar disorder or minor depressive disorder, are provided herein. The amount of the combination of agents (i.e., bupropion and (at least one) serotonin 5-HT_{1A} partial agonist, such as azapirones, preferably buspirone) is effective to treat the depressive disorder. It is important to note the synergistic effects of the combination of agents: even though one or more of the agents administered alone at a particular dosage may not be effective, when administered in combination, at the same dosage of each agent, the treatment is effective. The doses of the one or more of the agents in the combination therefore can be less than the FDA approved doses of each agent.

Particularly preferred methods include treating a depressive disorder by administering an effective amount of the combination of bupropion and buspirone to an individual having a depressive disorder.

### Dosages

The optimal dose of the combination of agents for treatment of a depressive disorder can be determined empirically for each individual using known methods and will depend upon a variety of factors, including the activity of the agents; the age, body weight, general health, gender and diet of the individual; the time and route of administration; and other medications the individual is taking. Optimal dosages may be established using routine testing and procedures that are well known in the art.

Typically, between about 10 mg/day to about 450 mg/day of bupropion will be administered to an individual in combination with the other agent(s) for treatment of a depressive disorder. Preferably the dose is 50-450 mg/day. More preferably, the dose is 100-200 mg/day.

Typically, between about 1 mg/twice per day to about 45 mg/twice per day of serotonin 5-HT_{1A} partial agonist, such as azapirones, preferably buspirone, will be administered to an individual in combination with the additional agent(s) for treatment of a depressive disorder. Preferably the dose is 2-45 mg/twice per day. More preferably, the dose is 5-15 mg/twice per day.

Smaller doses can be administered as appropriate (e.g., to children, the elderly, individuals with other medical conditions). The daily dose will vary from individual to individual and from time to time for a given individual (e.g., as daily dose is adjusted with the individual's changing mental states or general health). Smaller doses are likely to be used in pediatric individuals.

The amount of combination of agents that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, for treatment of most depressive disorders, a dosage regimen of 4 times daily or less is preferred. For the treatment of depressive disorders, including major depression, a dosage regimen of 1 or 2 times daily is particularly preferred. A single dose per day is most preferred. In certain embodiments, administration at meal times is preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

### Pharmaceutical Formulations and Routes of Administration

Provided herein are pharmaceutical formulations comprising a combination of agents for the treatment of a depressive disorder. The pharmaceutical formulations may additionally comprise a carrier or excipient, stabilizer, flavoring agent, and/or coloring agent.

Provided herein are pharmaceutical formulations comprising combination of agents which can be, for example, a combination of two types of agents: (1) bupropion and/or pharmacologically active metabolites, salts, solvates and racemates of bupropion and (2) at least one serotonin 5-HT_{1A} partial agonist, such as azapirones, preferably buspirone, and/or pharmacologically active metabolites, salts, solvates and racemates of serotonin 5-HT_{1A} partial agonist(s), such as azapirones, preferably buspirone. In a specific preferred embodiment, the combination of agents comprises bupropion and buspirone. In other embodiments, additional antidepressant agents may be added to the combination.

In some embodiments, it may be preferred to administer the combination of agents in a manner that suppresses formation of certain metabolites of one or more of the active agents in the combination. For example, U.S. Pat. No. 6,312,717 describes that formation of the 1-pyrimidinylpiperazine (1-PP) metabolite of buspirone can be reduced when the route of administration is selected from transmucosal, transdermal, or peroral administration using an extended release oral formulation. See U.S. Pat. No. 6,008,222 for another method of reducing formation of 1-PP.

The combination of agents may be administered using a variety of routes of administration known to those skilled in the art. Routes of administration include oral administration, preferably extended release, preferably once or twice per day. In certain preferred embodiments, a pharmaceutical formulation comprising a combination of agents may be taken orally in the form of liquid, syrup, tablet, capsule, powder, sprinkle, chewtab, or dissolvable disc. Alternatively, pharmaceutical formulations of the present invention can be administered intravenously or transdermally. Additional routes of administration known to those skilled in the art (see, e.g., Remington's Pharmaceutical Sciences, Gennaro A. R., Ed., 20^{th} Edition, Mack Publishing Co., Easton, Pa.).

### Dosage Forms: Release Properties

Buspirone is typically administered two times daily, while additional agents in the combination formulations provided herein, such as bupropion are administered once per day, twice per day, or three times per day. A combination formulation (such as the preferred combination of buspirone and bupropion) can be formulated in such a way that it is not necessary to administer buspirone separately between doses of the combination formulation. If the additional agent is administered once daily when given alone, the combination formulation containing buspirone and the additional agent can also be formulated for once daily administration. In certain embodiments the release properties of buspirone are modified to achieve this result.

The dosage forms useful in accordance with the invention can be characterized by the release properties of the formulation. The dosage forms can be immediate or modified release dosage forms in which the rate of release of the combination of agents in the blood stream is regulated. Sustained release formulations can be used to provide release over a period of time (e.g. one or more hours, several days or longer). Preferably, the sustained-release form avoids “dose dumping,” the production of a rapid rise and in the blood or plasma concentration of active agent, upon oral administration. The sustained-release oral dosage form can be formulated to provide for an increased duration of therapeutic action permitting effective once-daily dosing. Generally in a sustained-release dosage form the active agent release extends longer e.g., by several hours, than active agent release from the immediate-release dosage form. For example, sustained release forms of bupropion have been described in the prior art, e.g., U.S. Pat. Nos. 4,687,660, 5,358,970, 5,427,798, 6,033,686, 6,096,341 and 6,143,327. Sustained release forms of buspirone have been described in, e.g., U.S. Pat. Nos. 6,268,368, 6,500,459 and 6,893,661.

A sustained-release dosage form generally comprises a release-retarding material. The release-retarding material can be, for example, in the form of a matrix or a coating. An agent in sustained-release form may be, for example, a particle of the agent (e.g., buspirone and/or bupropion) that is combined with a release-retarding material. The release-retarding material is a material that permits release of active agent at a sustained rate in an aqueous medium. The release-retarding material can be selectively chosen so as to achieve, in combination with the other stated properties, a desired in vitro release rate. A wide variety of materials useful to produce a sustained release form is known to those of skill in the art. They may be in the form, for example, of a hydrophilic polymer, a hydrophobic polymer, a combination of hydrophilic and hydrophobic polymer and can be, for example, surfaces (beads, flat surfaces) onto which an agent is coated and/or materials into which an agent is incorporated or embedded (e.g., spheres, films or other appropriate embodiments or shape).

### Formulations

The combination pharmaceutical formulations provided herein may be formulated by a variety of methods apparent to those of skill in the art of pharmaceutical formulation. The various release properties described above may be achieved in a variety of different ways. Suitable formulations include, for example, tablets, capsules, press coat formulations, and other easily administered formulations.

The dosage form can be prepared by various conventional mixing, comminution and fabrication techniques readily apparent to those skilled in the chemistry of drug formulations.

### Examples of such techniques are as follows

(1) Direct compression, using appropriate punches and dies; the punches and dies are fitted to a suitable rotary tabletting press;

(2) Injection or compression molding using suitable molds fitted to a compression unit;

(3) Granulation followed by compression; and

(4) Extrusion in the form of a paste, into a mold or to an extrudate to be cut into lengths.

When particles are made by direct compression, the addition of lubricants may be helpful and sometimes important to promote powder flow and to prevent capping of the particle (breaking off of a portion of the particle) when the pressure is relieved. Useful lubricants are magnesium stearate (in a concentration of from 0.25% to 3% by weight, preferably less than 1% by weight, in the powder mix), and hydrogenated vegetable oil (preferably hydrogenated and refined triglycerides of stearic and palmitic acids at about 1% to 5% by weight, most preferably about 2% by weight). Additional excipients may be added to enhance powder flowability and reduce adherence.

### Preparation of Agent-Containing Subunits

The combination of agents may be prepared in a variety of ways, for example as subunits of a unit dosage form. Pellets comprising an active agent can be prepared, for example, by a melt pelletization technique. In this technique, the active agent in finely divided form is combined with a binder and other optional inert ingredients, and thereafter the mixture is pelletized, e.g., by mechanically working the mixture in a high shear mixer to form the pellets (e.g., pellets, granules, spheres, beads, etc., collectively referred to herein as “pellets”). Thereafter, the pellets can be sieved in order to obtain pellets of the requisite size. The binder material may also be in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like, and combinations comprising one or more of the foregoing binders.

Oral dosage forms may be prepared to include an effective amount of melt-extruded subunits containing the combination of agents in the form of multiparticles within a capsule. For example, a plurality of the melt-extruded muliparticulates can be placed in a gelatin capsule in an amount sufficient to provide an effective release dose when ingested and contacting by gastric fluid.

Subunits, e.g., in the form of multiparticulates, can be compressed into an oral tablet using conventional tabletting equipment using standard techniques. The tablet formulation may include excipients such as, for example, an inert diluent such as lactose, granulating and disintegrating agents such as cornstarch, binding agents such as starch, and lubricating agents such as magnesium stearate.

Alternatively, the subunits containing the combination of agents are added during the extrusion process and the extrudate can be shaped into tablets by methods know in the art. The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates can be any geometrical shape within this size range. Alternatively, the extrudate can simply be cut into desired lengths and divided into unit doses of active agent without the need of a spheronization step.

The melt-extruded dosage forms can further include combinations of melt-extruded multiparticulates containing one or more of the therapeutically active agents before being encapsulated. Furthermore, the dosage forms can also include an amount of one or more of the agents (e.g. buspirone) formulated for immediate-release for prompt therapeutic effect. The agent(s) formulated for immediate-release can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

A melt-extruded material may be prepared without the inclusion of subunits containing active agent, which are added thereafter to the extrudate. The mixture is then tabletted in order to provide release of the combination of agents. Such formulations can be particularly advantageous, for example, when an active agent included in the formulation is sensitive to temperatures needed for softening the hydrophobic material and/or the retardant material.

The oral dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

### Particles

Many of the oral dosage forms useful herein contain the combination of agents or individual agents of the combination of agents in the form of particles. Such particles may be compressed into a tablet, present in a core element of a coated dosage form, such as a taste-masked dosage form, a press coated dosage form, or an enteric coated dosage form, or may be contained in a capsule, osmotic pump dosage form, or other dosage form.

### Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Particular embodiments provided herein include capsules or tablets comprising buspirone and bupropion (or their metabolites). Appropriate amounts per dose of these agents individually are well known in the art, but the dosage forms useful in accordance with the invention can include smaller quantities of the agents due to the synergistic effects described herein.

### Press Coat Formulations

A press coat oral dosage form of the combination of agents comprises a core composition and a coating composition press-coated on the core. The core composition comprises a waxy material and active agent and the coating composition comprises a hydrophilic polymer and optionally active agent or a salt thereon.

The core composition of the press coat dosage from comprises a waxy material. The waxy material can be a hydrophobic waxy material to provide controlled-release of the combination of agents. In pharmaceutical products, for example, such waxy materials may be, for example, carnauba wax, tribehenin, fatty alcohols (particularly those having 12-24 carbon atoms, such as lauryl alcohol, myristyl alcohol, stearyl alcohol, palmityl alcohol, etc.), fatty acids (particularly those having 12-24 carbon atoms, such as lauric acid, myristic acid, stearic acid, palmitic acid, etc), polyethylenes, castor wax, C₁₆₋₃₀ fatty acid triglycerides, beeswax, and combinations comprising one or more of the foregoing waxes.

The coating composition comprises a hydrophilic polymer. The hydrophilic polymer can provide for controlled-release of active agent. The hydrophilic polymer providing controlled-release may be a film-forming polymer, such as a hydrophilic cellulose polymer. Such a hydrophilic cellulose polymer may be hydroxyalkyl cellulose polymer, for example hydroxyethylcellulose (HEC), hydroxypropyl cellulose (HPC), hydroxypropylmethylcellulose (HPMC), hydroxypropylethylcellulose (HPEC), hydroxypropylpropylcellulose (HPPC), hydroxypropylbutylcellulose (HPBC), and combinations comprising one or more of the foregoing polymers.

Both the core composition and the coating composition may further include a filler, such as a water insoluble filler, water soluble filler, and mixtures thereof.

Optional excipients can also be present in the core composition and the coating composition, including lubricants (such as talc and magnesium stearate), glidants (such as fumed or colloidal silica), pH modifiers (such as acids, bases and buffer systems), pharmaceutically useful processing aids, and combinations comprising one or more of the foregoing excipients. Excipients in the coating composition can be the same or different as those in the core composition.

In formation of the dosage form, the core composition can be press-coated with the press-coat composition coating formulation to form a tablet. The tablet can be further coated with optional additional coatings. The additional coatings can be pH-dependent or pH-independent, aesthetic or functional, and can include active agent in immediate or controlled-release. The additional coating may, for example, include an immediate-release dosage form of one or more of the individual agents of the combination of agents.

In forming the dosage form, the core composition components (active agent, wax, and optional excipients) are blended together and compressed into suitable cores. The blending can take place in a suitable order of addition. The cores may be blended by starting with the smallest volume component and then successively adding the larger volume components. Another process is to melt the wax and to blend active agent and optional excipients into the melted wax. Alternatively, active agent, wax and optional excipients can be blended together and then subjected to a temperature at which the wax will melt. Once cooled, the solidified mass can be milled into granules for compaction into cores.

Thus an embodiment of the invention pertains to a press-coat dosage form comprising a core composition comprising a combination of agents, a waxy material; and a coating composition comprising a hydrophilic polymer, wherein the coating composition is press-coated onto the core composition.

The invention also pertains to a press-coat dosage form comprising a core composition comprising the combination of agents, a waxy material; and a coating composition comprising a hydrophilic polymer, wherein the coating composition which also contains at least one additional active agent, is press-coated onto the core.

In certain embodiments the waxy material of the press-coat dosage form core is carnauba wax, tribehenin, fatty alcohols, lauryl alcohol, myristyl alcohol, stearyl alcohol, palmityl alcohol, fatty acids, lauric acid, myristic acid, stearic acid, palmitic acid, polyethylenes, castor wax, C₁₆₋₃₀ fatty acid triglycerides, beeswax, or any combination thereof. In some embodiments of the invention the hydrophilic polymer in the coating composition of the active agent press-coat dosage form comprises a hydrophilic cellulose polymer, preferably hydroxypropylmethyl cellulose (HPMC).

Yet another embodiment of the invention pertains to a combination press-coat dosage form comprising a core composition comprising the combination of agents and carnauba wax, a coating composition comprising one or more of the individual agents of the combination of agents and hydroxypropylmethyl cellulose (HPMC), wherein the coating composition is press-coated onto the core, and an additional coating composition comprising one or more of the individual agents of the combination of agents. In some embodiments of the invention the additional coating composition is an immediate-release coating composition.

### Easily Administered Dosage Forms

The invention provides easily administerable dosage forms for administration to patients who have difficulty swallowing, to reduce the risk of choking upon administration, and to improve patient compliance. Such dosage forms are particularly useful for administration to elderly and juvenile patients. The invention provides, for example, sprinkle dosage forms, liquid formulations, taste-masked liquid dosage forms and fast-dissolve dosage forms.

### Chewable Tablets

Another solid dosage form is a chewable tablet containing the combination of agents. A chewable tablet comprises a chewable base and optionally a sweetener. The chewable base can comprise an excipient such as, for example, mannitol, sorbitol, lactose, or a combination comprising one or more of the foregoing excipients. The optional sweetener used in the chewable dosage form may be, for example, digestible sugars, sucrose, liquid glucose, sorbitol, dextrose, isomalt, liquid maltitol, aspartame, lactose, and combinations comprising one or more of the foregoing sweeteners. In certain cases, the chewable base and the sweetener may be the same component.

The chewable dosage form may additionally contain preservatives, agents that prevent adhesion to oral cavity and crystallization of sugars, flavoring agents, souring agents, coloring agents, and combinations comprising one or more of the foregoing agents. Glycerin, lecithin, hydrogenated palm oil or glyceryl monostearate may be used as a protecting agent of crystallization of the sugars in an amount of about 0.04 to about 2.0 weight % of the total weight of the ingredients, to prevent adhesion to oral cavity and improve the soft property of the products. Additionally, isomalt or liquid maltitol may be used to enhance the chewing properties of the chewable dosage form.

### Fast Dissolving Dosage Forms

Another combination oral dosage form is a non-chewable, fast dissolving dosage form of the combination of agents. These dosage forms can be made by methods known to those of ordinary skill in the art of pharmaceutical formulations. For example, Cima Labs has produced oral dosage forms including microparticles and effervescents which rapidly disintegrate in the mouth and provide adequate taste-masking. Cima Labs has also produced a rapidly dissolving dosage form containing active agent and a matrix that includes a nondirect compression filler and a lubricant. Fast-dissolving dosage forms are disclosed in U.S. Pat. No. 5,178,878 and U.S. Pat. No. 6,221,392, which are hereby incorporated by reference for their teachings regarding fast-dissolve dosage forms.

### Optional Additional Additives for Combination Formulations

### Excipients

Excipients useful in the combination formulations include inert substances used as a diluent or vehicle for the combination of agents. Excipients may be added to facilitate manufacture, enhance stability, control release, enhance product characteristics, enhance bioavailability, enhance patient acceptability, etc. Pharmaceutical excipients include binders, disintegrants, lubricants, glidants, compression aids, colors, sweeteners, preservatives, suspending agents, dispersing agents, film formers, flavors, printing inks, etc. Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders. Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. KOLLIDON CL and POLYPLASDONE XL; cross-linked sodium carboxymethylcellulose (sodium croscarmellose), e.g. AC-DI-SOL, PRIMELLOSE; sodium starch glycolate, e.g. PRIMOJEL; sodium carboxymethylcellulose, e.g. NYMCEL ZSB10; sodium carboxymethyl starch, e.g. EXPLOTAB; ion-exchange resins, e.g. DOWEX or AMBERLITE; microcrystalline cellulose, e.g. AVICEL; starches and pregelatinized starch, e.g. STARCH 1500, SEPISTAB ST200; formalin-casein, e.g. PLAS-VITA, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide.

### Fillers

Formulations can also contain a filler, such as a water insoluble filler, water soluble filler, and combinations thereof. The filler may be a water insoluble filler, such as silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, preferably lactose, glucose, fructose, sucrose, mannose, dextrose, galactose, the corresponding sugar alcohols and other sugar alcohols, such as mannitol, sorbitol, xylitol, and combinations comprising one or more of the foregoing fillers.

### Coatings

The formulations described herein may be coated with a functional or non-functional coating. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol poly (ethylene oxide), poly (ethylene terephthalate), poly(vinyl alcohol), poly(vinyl isobutyl ether), poly(vinyl acetate), poly (vinyl chloride), polyvinyl pyrrolidone, and combinations comprising one or more of the foregoing polymers.

In applications such as taste-masking, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and the like, and combinations comprising one or more of the foregoing polymers.

In controlled-release applications, for example, the coating can be a hydrophobic polymer that modifies the release properties of active agent from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 to about 50 percent by weight of the polymer. Concentrations of the plasticizer, however, can be determined by routine experimentation.

### Packaged Formulations

Packaged pharmaceutical formulations or pharmaceutical products are included herein. Such packaged formulations include one or more pharmaceutical formulations comprising a combination of bupropion and (at least one) serotonin 5-HT_{1A} partial agonist, such as azapirones, preferably buspirone. Pharmacologically active metabolites, salts, solvates and racemates of bupropion and the (at least one) serotonin 5-HT_{1A} partial agonist, such as azapirones, preferably buspirone, also can be used pharmaceutical formulations of the invention. The combination of agents in formulated form is contained in a container; the package typically also contains instructions for using the formulation to treat an animal (typically a human patient) suffering from a depressive disorder.

In certain embodiments the packaged pharmaceutical formulation or pharmaceutical product contains the combination of agents described herein in a container with instructions for administering the dosage forms on a fixed schedule. In some of these embodiments, the combination of agents is provided in separate unit dosage forms.

It is preferred that the agents of the combination be dosed on the same schedule, whether by administering a single formulation or unit dosage form containing all of the agents of the combination, or by administering separate formulations or unit dosage forms of the agents of the combination. However, some of the agents used in the combination may be administered more frequently than once per day, or with different frequencies that other agents in the combination. Therefore, in one embodiment the packaged pharmaceutical formation contains a formulation or unit dosage form containing all of the agents in the combination of agents, and an additional formulation or unit dosage form that includes one of the agents in the combination of agents, with no additional active agent, in a container, with instructions for administering the dosage forms on a fixed schedule. For example, buspirone is frequently administered twice per day, while bupropion is frequently administered once per day (e.g., WELLBUTRIN XL). Thus an exemplary packaged pharmaceutical product includes one or more unit dosage forms that contain bupropion and buspirone, and additional one or more unit dosage forms that contain only buspirone. In this example, a complete daily dose would consist of one of each of the two different unit dosage forms, such as a tablet containing bupropion and buspirone, and a tablet containing only buspirone.

The invention includes providing prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

### EXAMPLES

The following examples further illustrate the invention but are not intended to limiting its scope in any way.

### Example 1

### Treatment of Depression with a Combination of Buspirone and Bupropion

The serotonin 5-hydroxytryptamine 1A (5-HT_{1A}) receptor partial agonist buspirone has effects on serotonergic systems, including presynaptic and postsynaptic receptors, that predict both anxiolytic and antidepressant activity [1]. Chronic administration of buspirone produces a downregulation of 5-HT₂ receptors, a finding common to most antidepressant drugs irrespective of mechanism of action [1]. The therapeutic effects of buspirone have been assessed in placebo-controlled studies of patients with major depression, and findings in these studies support antidepressant efficacy in addition to antianxiety effects [1], although buspirone is approved by the FDA only for the treatment of anxiety. Buspirone has also been shown to be a weak 5-HT_{2C} receptor antagonist, with a low affinity for 5-HT_{2A} and 5-HT_{2C} receptors [2]. The inhibitory influence of buspirone upon resting and fluoxetine-stimulated serotonin levels appears to reflect its agonist properties at serotonin 5-HT_{1A} autoreceptors [3]. However, it has been argued that the principal mechanism of action with respect to its antidepressant effects is probably the α₂-adrenergic antagonist properties of its metabolite, 1-(2-pyrimidinyl-piperazine) [3]. In a number of open-label studies [4] and one large randomized study [5], buspirone has shown to augment the antidepressant efficacy of selective serotonin reuptake inhibitors (SSRIs). Buspirone is thought to enhance the activity of SSRIs through the 5-HT_{1A} receptors [6].

Bupropion appears to produce antidepressant effects by blocking the reuptake of dopamine and norepinephrine, and has been in fact regarded as a norepinephrine dopamine reuptake inhibitor [7]. Bupropion, which is indicated by the FDA for the treatment of major depressive disorder (MDD), has been shown to enhance the antidepressant efficacy of serotonergic drugs such as the SSRIs in a number of open-label trials [4], but also in a recent, large randomized study [5].

Although, to applicant's knowledge, there are no published studies of the combination of buspirone and bupropion in the literature, one may hypothesize that the pharmacological effects of buspirone on the serotonergic and noradrenergic systems may lead to synergistic antidepressant effects when added to the effects of bupropion on dopamine and norepinephrine, by simultaneously affecting three distinct neurotransmitter systems (serotonin, norepinephrine and dopamine). Applicant reports herewith a clinical case where such synergy is suggested in the treatment of MDD.

### Case Report

A Caucasian woman in her late forties with a history of recurrent episodes of MDD presented with a recurrence of her MDD in the context of her having been off antidepressant medications. Her previous episodes had responded well to the combination of SSRIs (e.g. paroxetine 20 mg q.d.) and bupropion 150 mg q.d. sustained release (SR). Because of concerns about possible side effects (e.g. weight gain) related to SSRI treatment, the patient was started on bupropion 150 mg SR and buspirone 10 mg q.a.m. At the time of the initiation of this treatment combination, the patient reported depressed mood, irritability, decreased sleep and appetite, and fatigue, as well as reduced interest. She had a rapid response to this treatment combination, with marked symptomatic improvement across all domains except for insomnia. Because of worsening of her insomnia, the patient discontinued the buspirone while remaining on bupropion 150 SR q.d. This led to an improvement of her insomnia, but her depressive symptoms quickly returned.

### Discussion

This case report suggests that two agents often used for augmentation of SSRI treatment, buspirone and bupropion, may be effective when used together for the treatment of depression. The fact that this patient had a return of depressive symptoms upon discontinuation of the buspirone is suggestive of a possible synergistic effect of these two agents, and that treatment with bupropion 150 mg q.d. SR alone was not sufficient to lead to antidepressant response in this particular patient. One may hypothesize that such a synergy is due to the fact that these two agents, when used concomitantly, may affect three neurotransmitter systems (serotonin, norepinephrine and dopamine). Given the fact that previous reports have suggested that broadening the effect on neurotransmitter systems (such as when combining a noradrenergic drug with a serotonergic drug) may be more effective than using each agent alone with a predominant effect on one neurotransmitter [8], one may argue that combining bupropion and buspirone could lead to antidepressant effect synergy. Although it is possible that the synergy of these two agents may be mediated by some other pharmacological effects, the known effects of these two agents and of their active metabolites on the monoamine systems may certainly partly explain the potential benefit of such a combination. One may also speculate that the combination of bupropion and serotonin 5-HT_{1A} receptor partial agonist azapirones such as buspirone may be useful in the treatment of SSRI-resistant depressed patients or in major depressive disorder with or without anxious features, given the known antianxiety properties of buspirone. A theoretical advantage of such a combination for the treatment of depression may be that these types of agents have not been typically associated with weight gain and sexual dysfunction, two common side effects of SSRIs [9]. In fact, they have both been used to counteract side effects such as sexual dysfunction with SSRIs [10,11].

### REFERENCES

1. Robinson D S, Alms D R, Shrotriya R C, Messina M, Wickramaratne P: Serotonergic anxiolytics and treatment of depression. Psychopathology 1989; 22(suppl 1):27-36.
2. Assie M B, Lomenech H, Ravailhe V, Faucillon V, Newman-Tancredi A: Rapid desensitization of somatodendritic 5-HT_{1A} receptors by chronic administration of the high-efficacy 5-HT_{1A} agonist, F13714: a microdialysis study in the rat. Br J Pharmacol 2006; 149: 170-178.
3. Gobert A, Rivet J M, Cistarelli L, Melon C, Millan M J: Buspirone modulates basal and fluoxetine-stimulated dialysate levels of dopamine, noradrenaline and serotonin in the frontal cortex of freely moving rats: activation of serotonin 1A receptors and blockade of 2-adrenergic receptors underlie its actions. Neuroscience 1999; 93: 1251-1262.
4. Fava M, Rush A J: Current status of augmentation and combination treatments for major depressive disorder: a literature review and a proposal for a novel approach to improve practice. Psychother Psychosom 2006; 75: 139-153.
5. Trivedi M H, Fava M, Wisniewski S R, Thase M E, Quitkin F, Warden D, Ritz L, Nierenberg A A, Lebowitz B D, Biggs M M, Luther J F, Shores-Wilson K, Rush A J, STAR*D Study Team Medication augmentation after the failure of SSRIs for depression. N Engl J Med 2006; 354: 1243-1252.
6. Redrobe J P, Bourin M: Dose-dependent influence of buspirone on the activities of selective serotonin reuptake inhibitors in the mouse forced swimming test. Psychopharmacology (Berl) 1998; 138: 198-206.
7. Fava M, Rush A J, Thase M E, Clayton A, Stahl S M, Pradko J F, Johnston J A: 15 years of clinical experience with bupropion HCl: from bupropion to bupropion SR to bupropion XL. Prim Care Companion J Clin Psychiatry 2005; 7: 106-113.
8. Nelson J C, Mazure C M, Jatlow P I, Bowers M B Jr, Price L H: Combining norepinephrine and serotonin reuptake inhibition mechanisms for treatment of depression: a double-blind, randomized study. Biol Psychiatry 2004; 55: 296-300.
9. Cassano P, Fava M: Tolerability issues during long-term treatment with antidepressants. Ann Clin Psychiatry 2004; 16: 15-25.
10. Clayton A H, Warnock J K, Kornstein S G, Pinkerton R, Sheldon-Keller A, McGarvey E L: A placebo-controlled trial of bupropion SR as an antidote for selective serotonin reuptake inhibitor-induced sexual dysfunction. J Clin Psychiatry 2004; 65: 62-67.
11. Landen M, Eriksson E, Agren H, Fahlen T: Effect of buspirone on sexual dysfunction in depressed patients treated with selective serotonin reuptake inhibitors. J Clin Psychopharmacol 1999; 19:268-271.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All references disclosed herein are incorporated by reference, in particular for the teaching that is referenced hereinabove.

1. A method of treating a depressive disorder in an individual comprising
administering to the individual an amount of (1) bupropion and/or a salt, solvate, metabolite or racemate thereof, and an amount of (2) at least one serotonin 5-HT_{1A} partial agonist and/or a salt, solvate, metabolite or racemate thereof, wherein the combined amount of (1) and the amount of (2) is effective for treatment of the depressive disorder.

2. The method of claim 1, wherein the serotonin 5-HT_{1A} partial agonist is an azapirone and/or a salt, solvate, metabolite or racemate thereof.

3. The method of claim 1, wherein the azapirone is buspirone, gepirone, ipsapirone, tandospirone or zalospirone, and/or a salt, solvate, metabolite or racemate thereof.

4. The method of claim 3, wherein the azapirone is buspirone and/or a salt, solvate, metabolite or racemate thereof.

5.-6. (canceled)

7. The method of claim 1, wherein the depressive disorder is major depressive disorder, dysthymic disorder, minor depressive disorder, or bipolar disorder.

8. (canceled)

9. The method of claim 1, wherein the treatment comprises co-administering the amount of (1) and the amount of (2).

10. The method of claim 9, wherein the amount of (1) and the amount of (2) are in a single formulation or unit dosage form.

11.-12. (canceled)

13. The method of claim 9, wherein the amount of (1) and the amount of (2) are in a separate formulations or unit dosage forms.

14. The method of claim 1, wherein the treatment comprises administering the amount of (1) and the amount of (2) at substantially the same time.

15. The method of claim 1, wherein the treatment comprises administering the amount of (1) and the amount of (2) at different times.

16. The method of claim 1, wherein the amount of (1) and/or the amount of (2) is administered at dosages that would not be effective when one or both of (1) and (2) is administered alone, but which amounts are effective in combination.

17. A pharmaceutical formulation comprising an amount of (1) bupropion and/or a salt, solvate, metabolite or racemate thereof, and an amount of (2) at least one serotonin 5-HT_{1A} partial agonist and/or a salt, solvate, metabolite or racemate thereof, wherein the combined amount of (1) and (2) is effective for treatment of depressive disorder.

18. The pharmaceutical formulation of claim 17, wherein the serotonin 5-HT_{1A} partial agonist is an azapirone and/or a salt, solvate, metabolite or racemate thereof.

19. The pharmaceutical formulation of claim 17, wherein the azapirone is buspirone, gepirone, ipsapirone, tandospirone or zalospirone, and/or a salt, solvate, metabolite or racemate thereof.

20. The pharmaceutical formulation of claim 17, wherein the azapirone is buspirone and/or a salt, solvate, metabolite or racemate thereof.

21. (canceled)

22. The pharmaceutical formulation of claim 17, wherein the amount of (1) and the amount of (2) are in a single formulation or unit dosage form.

23. The pharmaceutical formulation of claim 17, wherein the formulation or unit dosage form is an oral formulation or unit dosage form.

24. (canceled)

25. The pharmaceutical formulation of claim 17, wherein the amount of (1) and/or the amount of (2) would not be effective when one or both of (1) and (2) is administered alone, but which amounts are effective in combination.

26. A pharmaceutical product comprising
a first formulation or unit dosage form comprising an amount of (1) bupropion and/or a salt, solvate, metabolite or racemate thereof, and an amount of (2) at least one serotonin 5-HT_{1A} partial agonist and/or a salt, solvate, metabolite or racemate thereof, and
a second formulation or unit dosage form comprising an amount of (1) but not (2) or an amount of (2) but not (1),
wherein the combination of the first formulation or unit dosage form and the second formulation or unit dosage form is effective for treatment of a depressive disorder.

27. A pharmaceutical product comprising
a first formulation or unit dosage form comprising an amount of bupropion and/or a salt, solvate, metabolite or racemate thereof, and
a second formulation or unit dosage form comprising an amount of at least one serotonin 5-HT_{1A} partial agonist and/or a salt, solvate, metabolite or racemate thereof,
wherein the combination of the first formulation or unit dosage form and the second formulation or unit dosage form is effective for treatment of a depressive disorder.

28. The pharmaceutical product of claim 26, wherein the serotonin 5-HT_{1A} partial agonist is an azapirone and/or a salt, solvate, metabolite or racemate thereof.

29. The pharmaceutical product of claim 26, wherein the azapirone is buspirone, gepirone, ipsapirone, tandospirone or zalospirone, and/or a salt, solvate, metabolite or racemate thereof.

30. The pharmaceutical product of claim 26, wherein the azapirone is buspirone and/or a salt, solvate, metabolite or racemate thereof.

31. (canceled)

32. The pharmaceutical product of claim 26, wherein the formulation or unit dosage form is an oral formulation or unit dosage form.

33. (canceled)

34. The pharmaceutical product of claim 26, wherein the amount of (1) and/or the amount of (2) would not be effective when one or both of (1) and (2) is administered alone, but which amounts are effective in combination.
